(19) United States
(12) Patent Application Publication
(10) Pub. No.: US 2012/0080037 A1
(43) Pub. Date: Apr. 5, 2012

(54) NASOPHARYNGEAL AIRWAY

(75) Inventors: Bahman Guyuron, Lyndhurst, OH (US); Jamie Horvath, Twinsburg, OH (US)

(73) Assignee: INNOVATIVE MEDICAL EQUIPMENT, LLC, Lyndhurst, OH (US)

(21) Appl. No.: 12/898,056

(22) Filed: Oct. 5, 2010

(51) Int. Cl. A61M 16/00

(52) U.S. Cl. 128/207.18

(57) ABSTRACT

A nasopharyngeal airway for providing an air passage through a nose to a nasopharynx includes a cannula having a proximal open end, a distal closed end, and a sidewall surrounding a lumen of the cannula. The nasopharyngeal airway has an opening in the sidewall at a distal portion of the cannula for providing fluid communication between a distal portion of the lumen and the nasopharynx when the cannula is inserted into the nose and a conduit in the sidewall of the cannula, the conduit having an opening adjacent to the proximal open end of the cannula and a conduit opening in a distal portion of the cannula for providing fluid communication between the conduit and the distal portion of the lumen.

### TECHNICAL FIELD

The present invention generally relates to an airway adjunct, and more particularly to a nasopharyngeal airway.

### BACKGROUND OF THE INVENTION

A nasopharyngeal airway, also referred to as a nasal trumpet, is a device that is inserted into a patient's nose to maintain an open airway through the nasal passage to the patient's nasopharynx. The nasopharyngeal airway can reduce or eliminate airway collapse and also may minimize or reduce the possibility that a patient's tongue may slide back in the patient's throat to obstruct the airway. Nasopharyngeal airways commonly are used for patients who are undergoing procedures while under sedation or who are awakening from general anesthesia. Emergency response personnel, such as paramedics, also may use a nasopharyngeal airway for patients who have experienced severe skull or facial trauma, for example, as a result of an accident.

In use, the nasopharyngeal airway is slid through the nostril, along the hard palate (e.g., the bony partition between the oral and nasal cavities) and into the nasopharynx. Inserting and maintaining the nasopharyngeal airway into a patient's nose may cause pain and discomfort for the patient.

Conventional nasopharyngeal airways consist of a tubular member (also referred to as a cannula) having a circular cross-section. The cannula has two ends, one for insertion into the patient's nose to the nasopharynx and the other for remaining external to the patient. The cannula surrounds a lumen that provides an open pathway to the nasopharynx. Tubes or medical instruments (for example, such as a carbon dioxide detector, an oxygen supply tube, a suction tube, etc.) can be inserted into the lumen or externally attached to the cannula. When located inside the lumen, these items consume part of the airway and therefore effectively reduce airflow to the nasopharynx. When located external to the cannula, these items may increase the size of the cannula and thus may make the nasopharyngeal airway more uncomfortable for the patient.

### SUMMARY

The present invention provides a nasopharyngeal airway having an oval contour and a soft, tapered, rounded tip to facilitate insertion of the nasopharyngeal airway into the nasal passage to reduce patient trauma and/or discomfort. The nasopharyngeal airway includes a cannula surrounding a main lumen extending from an open proximal end of the nasopharyngeal airway to the rounded tip. The nasopharyngeal airway also has one or more conduits embedded in the sidewall of the cannula extending from the open proximal end and opening to a distal portion of the lumen. The conduits may be embedded into the sidewall by integrally forming the conduit within the sidewall or by inserting the conduit into a channel in the sidewall. By embedding the conduits into the sidewall of the cannula, the conduits do not consume a substantial portion of the lumen. Additionally, complications arising from clogging, tangling, and/or collapsing of the conduits generally can be avoided.

The cross-section of the cannula can be ovular or elliptical in shape to conform to the shape of the nostril and to further facilitate the insertion of the cannula into the nasal passage and maximize usage of the space in the nasal passage. The nasal passage between the septum and turbinates is not round and the force that converts a round lumen to an oval lumen causes discomfort in the septum and turbinates. This forceful reshaping of the nasal trumpet can be avoided or reduced by this is oval or elliptical design of the nasal trumpet.

According to one embodiment, the nasopharyngeal airway includes a cannula having a proximal open end, a distal closed end, and a sidewall surrounding a lumen of the cannula. The sidewall has an opening for providing fluid communication between a distal portion of the lumen and the nasopharynx when the cannula is inserted into the nose. The sidewall has one or more conduits, each having an opening adjacent to the proximal open end of the cannula and an opening in a distal portion of the cannula for providing fluid communication between the conduit and the distal portion of the lumen. The conduit openings can be elongate in shape so as to reduce or minimize the effects of obstructions, such as mucus or debris.

According to another aspect, the nasopharyngeal airway includes a tubular member having a closed distal end, a sidewall surrounding a lumen, and an opening in the sidewall for providing fluid communication between a distal portion of the lumen and a nasopharynx of a person when the tubular member is inserted into a nose. The tubular member is tapered from the opening in the sidewall to the closed distal end to facilitate the insertion of the tubular member into the nose. The nasopharyngeal airway also includes a pair of parallel conduits in the sidewall of the tubular member, each conduit in fluid communication with the distal portion of the lumen through respective conduit openings.

According to another embodiment, a tip for a nasopharyngeal airway includes a tubular member having a sidewall surrounding a lumen and a pair of conduit openings in the sidewall of the tubular member. Each of the conduits is in fluid communication with the lumen through the respective openings. A pair of sidewall openings in the sidewall of the tubular member provides fluid communication between the lumen and a nasopharynx of a person when the tubular member is inserted into a nose, with the conduit openings and the sidewall openings being circumferentially offset from one another.

To the accomplishment of the foregoing and related ends, certain features described hereinafter are particularly pointed out in the claims. The following description and the annexed drawings set forth in detail certain illustrative embodiments. These embodiments, however, are merely indicative of a few of the various ways in which inventive features may be employed. Other objects, advantages and novel features will become apparent from the following detailed description when considered in conjunction with the drawings.

### BRIEF DESCRIPTION OF THE DRAWINGS

In the accompanying drawings, which are not necessarily drawn to scale:

FIG. 1 is a schematic representation of a nasopharyngeal airway inserted into a patient's nasal passage.

FIG. 2 is an isometric view of an exemplary nasopharyngeal airway.

FIG. 3 is a side view of the nasopharyngeal airway of FIG. 2.

FIG. 4 is a cross-sectional view of the nasopharyngeal airway of FIG. 3 at section lines 4-4.

FIG. 5 is a cross-sectional view of the nasopharyngeal airway of FIG. 3 at section lines 5-5.

FIG. 6 is an isometric view of the closed distal end of the nasopharyngeal airway of FIG. 1.

FIG. 7 is a sectional view of the closed distal end of the nasopharyngeal airway of FIG. 4.

FIG. 8 is a cross-sectional view of the closed distal end taken along section lines 8-8 of FIG. 7.

FIG. 9 is a cross-sectional view of the closed distal end taken along section lines 9-9 of FIG. 7.

FIG. 10 is an isometric view of a nasopharyngeal airway.

FIG. 11 is a side view of the nasopharyngeal airway of FIG. 10.

FIG. 12 is a cross-section view of the proximal end of the nasopharyngeal airway taken along section lines 12-12 of FIG. 11.

FIG. 13 is a back view of the nasopharyngeal airway of FIG. 10.

FIG. 14A is a cross-sectional view taken along section lines 14-14 of FIG. 10 without conduits in the channels.

FIG. 14B is a cross-sectional view taken along section lines 14-14 of FIG. 10 with conduits embedded in the channels.

FIG. 15 is an isometric view of the closed distal end of the nasopharyngeal airway of FIG. 10.

FIG. 16 is another isometric view of the closed distal end of the nasopharyngeal airway of FIG. 10.

FIG. 17 is cross-sectional view of the closed distal end of the nasopharyngeal airway taken along section lines 17-17 in FIG. 16.

### DETAILED DESCRIPTION

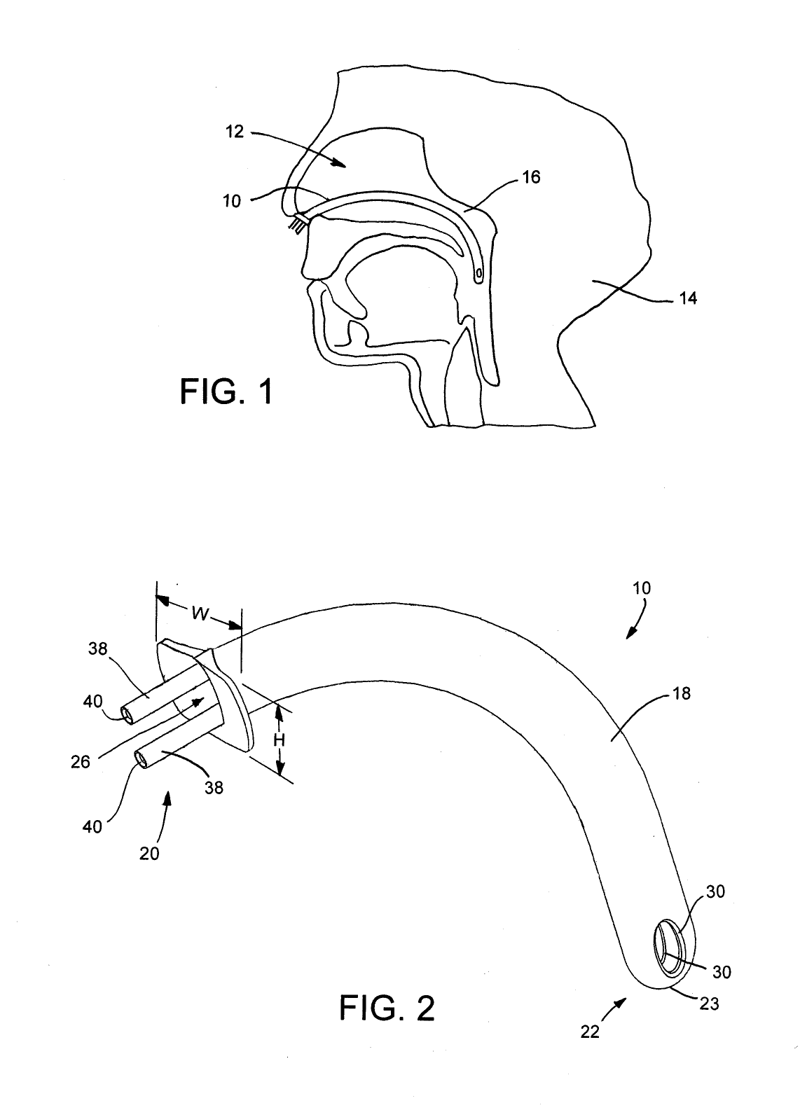

Referring to the drawings, wherein like reference numbers designate like parts in the several figures, and initially to FIG. 1, a schematic representation of a nasopharyngeal airway 10 inserted into a nasal passage 12 of a patient 14 is shown. The nasopharyngeal airway 10 secures an open airway through the patient's nasal passage 12 to the patient's nasopharynx 16.

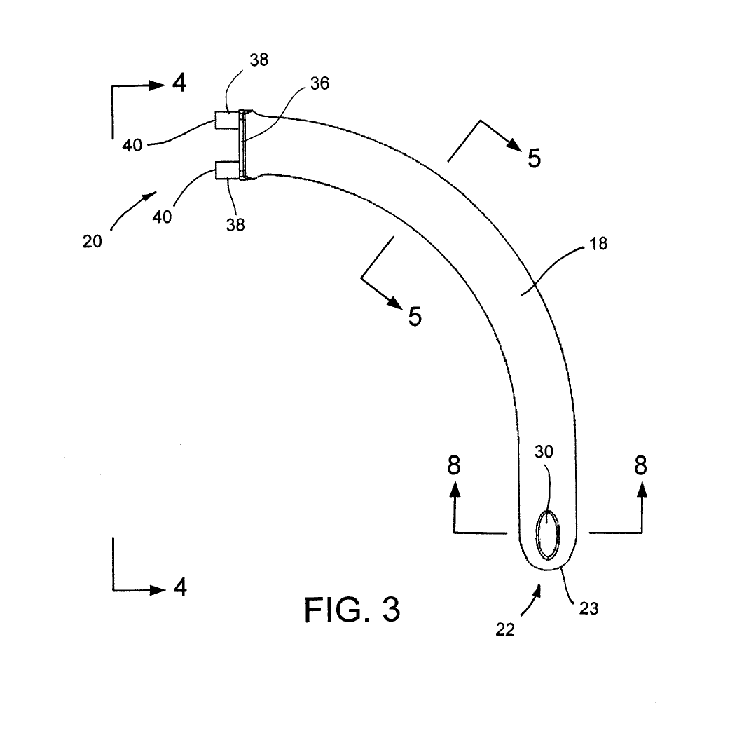

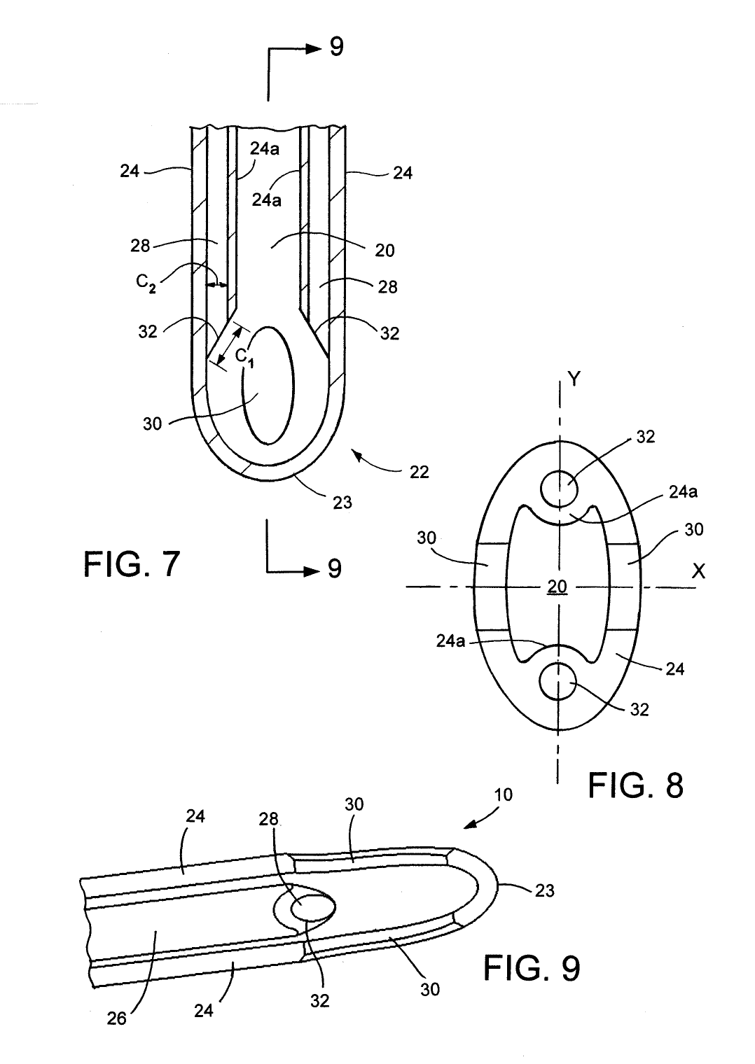

An exemplary embodiment of the nasopharyngeal airway 10 is shown in FIGS. 2-9. The nasopharyngeal airway 10 is constructed or formed from a soft, flexible material. The flexible material facilitates the insertion of the nasopharyngeal airway 10 into the nose and through the nasal passage 12 by allowing the nasopharyngeal airway 10 to bend and flex as may be necessary to navigate through the internal anatomy of the patient's nasal passage 12, for example, the turbinate, the septum and the hard palate. Exemplary suitable materials include a biocompatible thermoplastic elastomer or a biocompatible thermoset silicone, for example. The nasopharyngeal airway can be formed with an injection molding process.

The nasopharyngeal airway 10 includes a cannula 18 having a flared open proximal end 20 and a closed distal end 22 having a rounded tip 23. As shown best in FIG. 5, the outer surface of the cannula 18 has an oval or elliptical cross-sectional shape. The shape of the outer surface of the cannula 18 has been designed to match (e.g., correspond to) the shape of the nostril opening. The ovular shape facilitates the insertion of the cannula 18 through the nostril and into the nasal passage 12. By conforming to the general shape of the nostril and the nasal passage 12, pressure applied by the cannula 18 to the turbinate bone and hard palate of the patient can be reduced making the nasopharyngeal airway 10 easier to insert and more comfortable for the patient 14. The cannula 18 also may be formed or molded to have a natural curve so as to conform to the nasal passage and throat of the patient.

The tip 23 of the distal closed end 22 is rounded. The tip 23, which is made from the same soft and flexible material as the cannula 18, reduces the likelihood that the distal end of the cannula 18 will catch or grab onto portions of the nasal passage 12 as the tip 23 is slid through the nasal passage 12 to the nasopharynx 16.

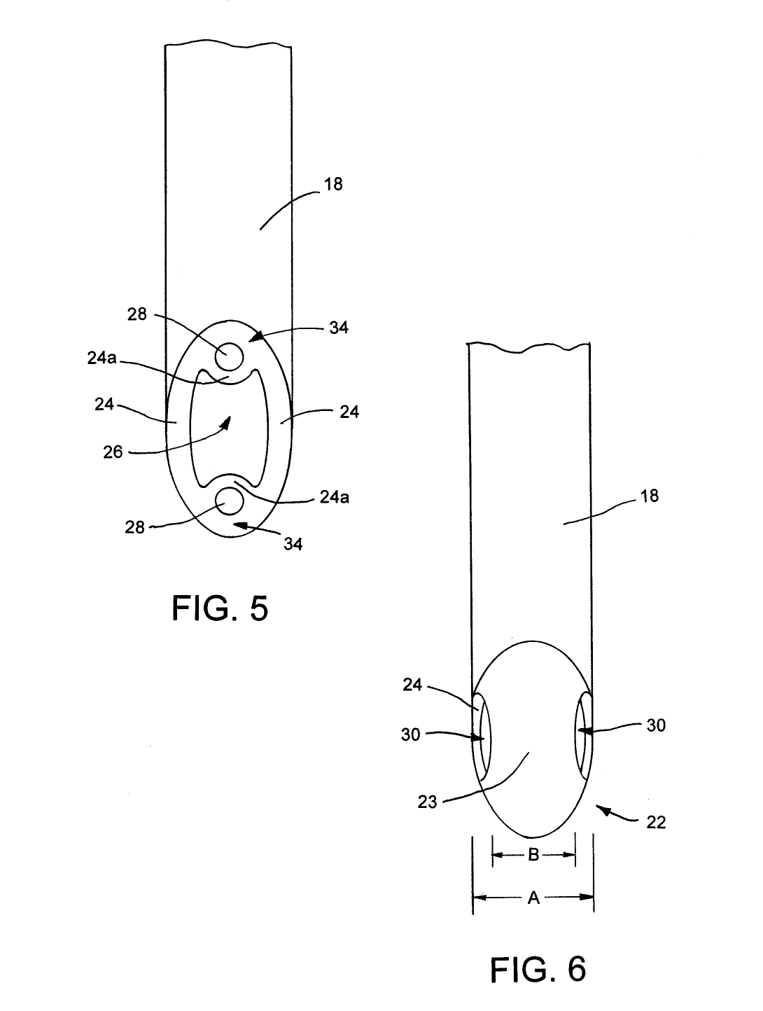

As shown best in FIG. 6, the closed distal end 22 of the cannula 18 also is tapered. For example, as shown in FIG. 6, the width A of the proximal portion of the tip 23 is wider than the width B of the distal end of the tip 23. The taper of the tip 23 facilitates the insertion of the cannula 18 into the nasal passage 12 by gradually expanding the nasal passage 12 as the cannula 18 is slid into the nostril and over the hard palate.

Thus, the tapered and the rounded tip 23 of the distal end 22 of the cannula 18 facilitate the atraumatic insertion of the cannula 18 into the nasal passage 12. In contrast, a cannula with an open end may be difficult to insert and may scratch the turbinate and septum of the patient and cause injury and/or bleeding.

The cannula 18 has a sidewall 24 that surrounds a lumen 26 through the nasopharyngeal airway 10. The lumen 26 may be tapered from the proximal end 20 to the distal end 22. The closed distal end 22 of the cannula 18 has one or more openings 30 in the sidewall 24 for providing fluid communication between the lumen 26 and the nasopharynx 16. In the exemplary embodiment of FIGS. 2-9, the distal portion of the nasopharyngeal airway has a pair of openings 30 in the sidewall 24, however, the nasopharyngeal airway may have a different number of openings, for example, only one opening or three openings, etc. The openings may be circular or ovular openings, or may have a different shape.

The multiple openings 30 in the sidewall 24 provide multiple pathways to the nasopharynx 16. In the event that one of the openings 30 becomes blocked, for example if the distal end 22 of the cannula 18 is pressed against the pharynx or against the tongue, air will still be able to pass through the lumen 26 to the nasopharynx 16 through another opening in the sidewall 24. In such a situation, the lumen 26 is not blocked, but rather remains open to allow for airflow to the nasopharynx 16 through one of the unblocked openings.

When slid into the nasal passage 12, the closed distal end 22 of the cannula 18 may rest on or against the tongue of the patient and/or the pharyngeal tissues in the patient's throat. The openings 30 in the sidewall of the cannula 18 therefore are less likely to be blocked by the tongue or the pharyngeal soft tissues since the closed distal end 22 is more likely to contact those structures rather than the openings 30 in the sidewall 24. The closed distal end 22 end protects (e.g., shields) the openings 30 and reduces the potential for blockage of the nasopharyngeal airway from the tongue and/or pharyngeal soft tissues.

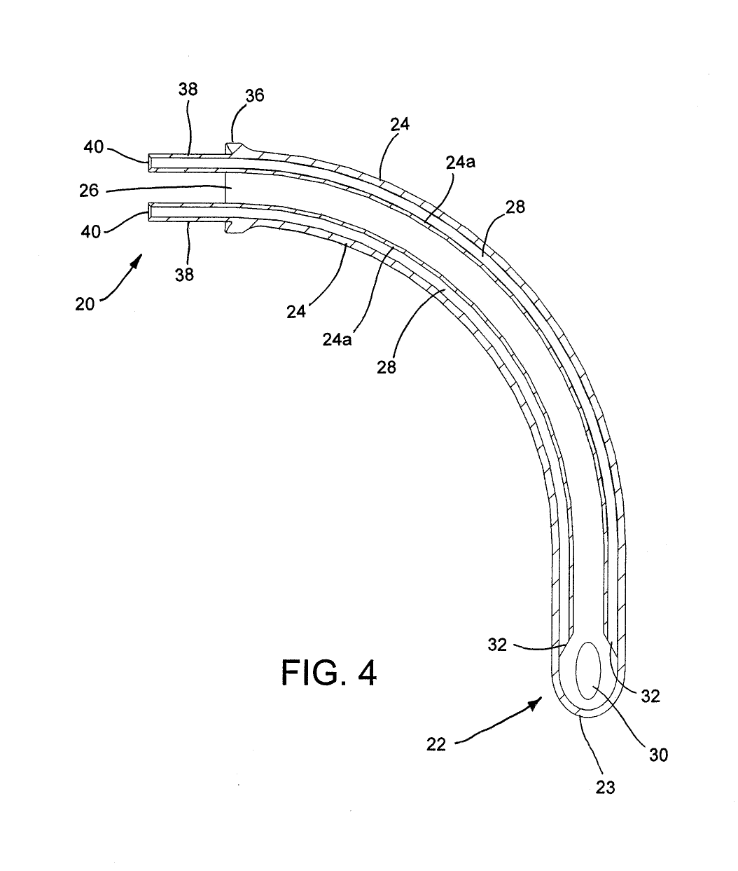

Embedded within the sidewall are one or more conduits 28. As shown in FIG. 4, the conduits 28 extend parallel to one another along a substantial portion of the length of the cannula 18. The conduits 28 extend the majority of the length of the cannula 18 and open to the main lumen 26 near the closed distal end 22. The conduits 28 are located in the sidewall 24 of the cannula 18 and are in fluid communication with the distal portion 22 of the lumen 26 via conduit openings 32.

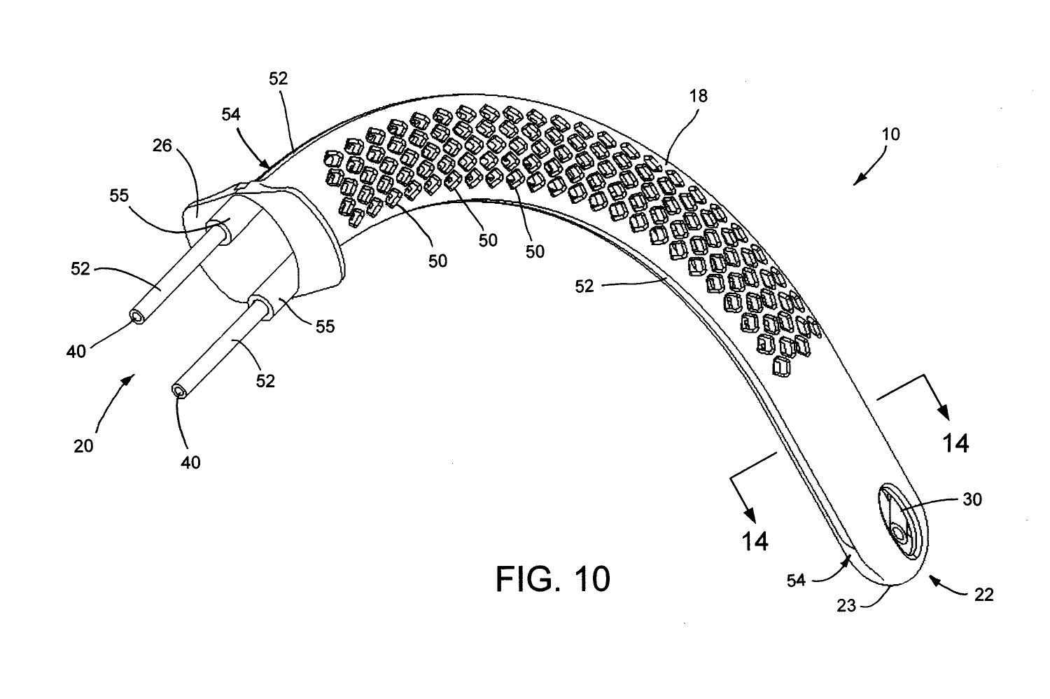

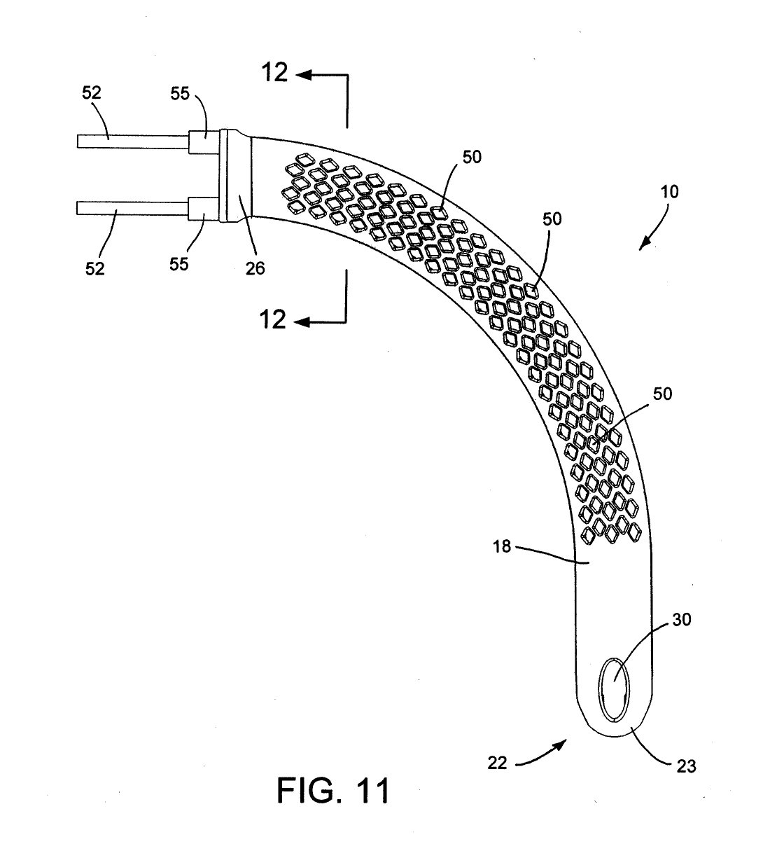

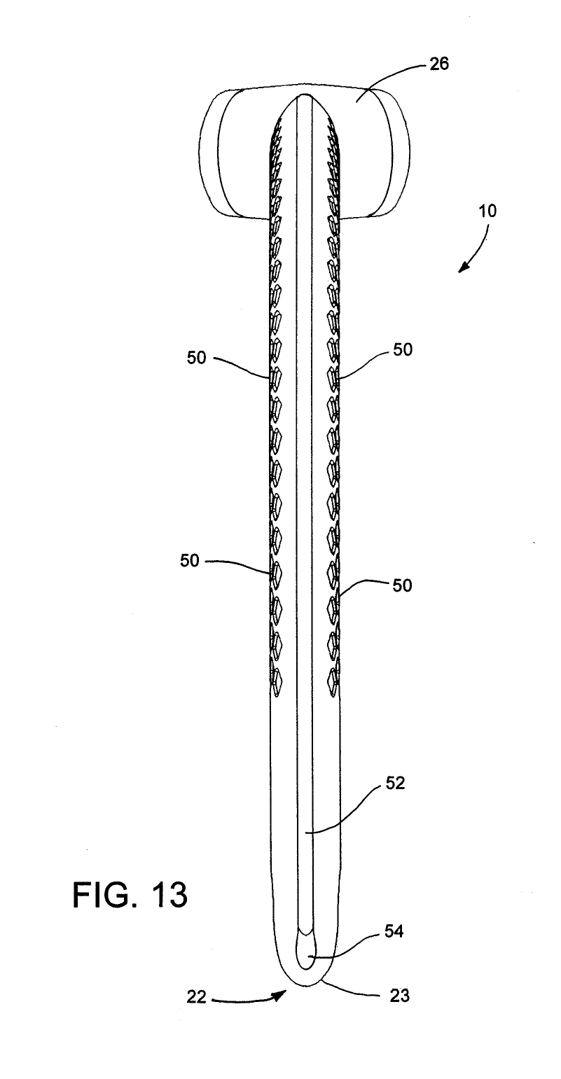

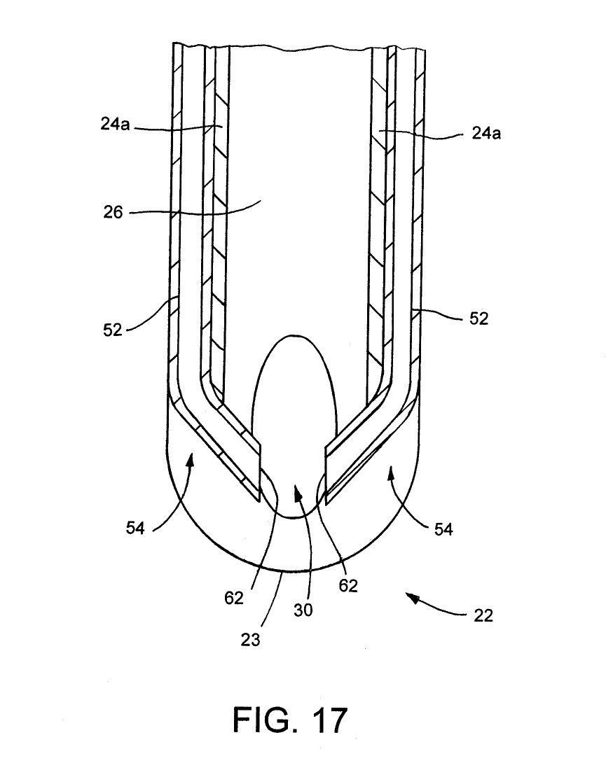

By embedding the conduits 28 in the sidewall of the cannula 18, the cannula takes advantage of the ovular shape of the nostril and utilizes most or all of the space in the nasal passage while also maximizing the internal width of the lumen 26. In the embodiment of FIGS. 2-9, two conduits 28 are integrally formed in the sidewall, however, other configurations are possible. For example, as described in more detail below with respect to FIGS. 10-17, the conduits may be at least partially contained within channels in the sidewall. The conduits can be inserted or otherwise placed in the channels by a secondary assembly process.

As shown best in FIG. 5, the conduits 28 can be integrally formed in the elongate portions 34 of the oval cross-section of the cannula 18. The conduits 28 are separated from the lumen 26 by internal portions 24a of the sidewall 24, and the internal portions 24a of the sidewall 24 may be curved around the conduits 28 so as to maximize the open space in the lumen 26. Embedding the conduits 28 in the sidewall 24 therefore optimizes the area of the lumen 26 to the nasopharynx 16 and also optimizes usage of the available space in the nostril.

Also shown in FIG. 5, each conduit 28 has a cross-sectional area that is less than the cross-sectional area of the lumen 26. For example, the cross-sectional area of the lumen may be about 0.05 in²-about 0.10 in² and preferably about 0.808 in², and the cross-sectional area of the each conduit may be about 0.001 in²-about 0.005 in² and preferably about 0.00283 in². The lumen 26 therefore provides a main pathway to the nasopharynx of the patient and the conduits provide secondary or smaller pathways that connect to the distal portion of the lumen.

In one embodiment, one of the conduits 28 can be connected to a gas supply (e.g., an oxygen supply) and the other conduit 28 can be used to measure CO₂ (e.g., by connecting the conduit 28 to a CO₂ sensor), and the gas can be delivered to the distal end portion 22 of the cannula 18 near the openings 30 in the sidewall 24. Delivering the gas to the distal portion of the cannula reduces the likelihood that the gas will accumulate near the proximal end of the cannula where it can be exposed to the external environment (e.g., the operating room or recovery room), which may be undesirable. For example, a spark or other ignition source in the environment could cause oxygen accumulated in a proximal portion of the cannula to ignite. This risk can be reduced by delivering the oxygen to the distal portion of the cannula 18. Additionally, the delivery of oxygen or another gas at a distal portion of the cannula can help to ensure that the patient is receiving the oxygen (via the openings 30 in the cannula) and that the oxygen is not being lost to the atmosphere through the open proximal end.

As mentioned, one of the conduits 28 may be coupled to a CO₂ sensor for sensing CO₂ to measure respiration. In the illustrated embodiment, the conduit 28 is in communication with the lumen 26 at the distal end portion 22 of the cannula 18, however, the conduit 28 may open to the lumen 26 at any location along the length of the lumen 26. For example, the conduit 28 may open to the lumen 26 at a location closer to the proximal end portion 20 of the cannula 18 rather than the distal end portion 22.

Opening the conduits 28 to the lumen 26 and terminating the conduits 28 near, but proximal to, the distal end 22 of the cannula 18 reduces the likelihood of the conduits 28 being blocked by debris and/or secretions in the nasal cavity because the conduit openings 32 are shielded and protected from direct interaction with the nasal passage 12. Additionally, as the cannula 18 is slid through the nasal passage 12, it is less likely that debris will interfere with the conduit openings 32 because the debris must first pass around the tip 23 of the closed distal end 22, into one of the openings 30 in the sidewall 24, and then into one of the conduit openings 32. Because the conduit openings 32 are less exposed to the interior of the nasal passage 12, they are less likely to become blocked by debris.

The locations of the openings 30 in the sidewalls 24 and the conduit openings 32 are shown in more detail by the cross-section of FIG. 8. As described above, the cannula 18 is generally elliptical or ovular in shape. The cross-section of the cannula 18 therefore has a minor axis X and a major axis Y. The sidewall openings 30 are located opposite to one another in the sidewall 24 at corresponding portions of the minor axis X, and the conduit openings 32 are located opposite to one another in the sidewall 24 at corresponding portions of the major axis Y. The sidewall openings 30 and the conduit openings 32 therefore are circumferentially (e.g., perpendicularly or angularly) offset from one another in the same portion of the cannula 18. The sidewall openings 30 and conduit openings 32 may at least partially overlap one another such that air may flow through the conduit openings directly through the sidewall openings to the nasopharynx.

Also shown in FIGS. 8 and 9, the cross-sectional area C₁ of each opening 32 from the conduit 28 to the lumen 26 is wider than the cross-sectional area C₂ of each conduit 28 proximal to the opening 32. This shape further reduces or minimizes the likelihood that the conduit openings 32 will become blocked by debris. The conduit openings 32 may be elongate in shape, which may further reduce the likelihood that the conduit openings 32 may be blocked by debris.

Referring back to FIG. 2, the proximal end 20 of the cannula 18 is flared and forms a flange 36. The flange 36 prevents the nasopharyngeal airway 10 from being inserted too far into the nose by engaging or abutting the sides of the nostrils when the cannula 18 is fully inserted into the nasal passage 12. The flange 36 therefore prevents the nasopharyngeal airway 10 from being inserted too deep into the patient's head such that it is difficult to remove or to a position where it may harm or otherwise injure the patient.

The flange 36 is elongate in shape and may, for example, have a width dimension W that is greater than a height dimension H. The flange 36 therefore may be similar in shape to a rectangle having rounded corners, as shown in FIG. 2. A rectangular flange is less likely than other shaped flanges, for example, a circular shaped flange, to engage or compress the upper lip of the patient when the nasopharyngeal airway is fully inserted into the nose. The rectangular shape also may more comfortable for the patient and less cumbersome for a physician to manipulate. The rectangular flange also may be less likely to interfere with the surgeon's work around the nose during surgery and/or when the patient is under sedation.

Also shown in FIG. 2, the conduits 28 may extend proximally from the flange 36, for example, as indicated generally by 38. The ends of the conduits 40 can be connected to external tubes, for example, the oxygen supply and carbon dioxide sensor tubes described above.

Referring now to FIGS. 10-17, another embodiment of a nasopharyngeal airway 10 is shown. Many portions of the nasopharyngeal airway of FIGS. 10-17 are the same as those described above with respect to FIGS. 2-9 and for purposes of brevity those portions will not be repeated below.

During normal respiration, air that is drawn into the lungs is naturally heated and moisturized by turbinates in the nasal passage. In some circumstances, it may be desirable to maintain the natural warming and physiologic humidification of air flow through the lumen 26 into the lungs of the patient when a nasopharyngeal airway is inserted into the nasal passage. The nasopharyngeal airway 10 of FIGS. 10-17 includes a plurality of holes 50 located so as to be generally aligned with and/or facing the turbinates and/or septum of the patient when the nasopharyngeal airway is inserted into the nasal passage. The holes 50 allow the air in the lumen 26 to be exposed to the turbinates and/or septum, allowing for the physiologic humidification and warming of the air flow into the lungs.

The holes 50 may facilitate the insertion of the nasopharyngeal airway into the nasal passage by increasing the flexibility of the nasopharyngeal airway. For example, the holes can provide for increased flexion and bending of the sidewall 24 of the cannula 18, thereby facilitating the insertion of the nasopharyngeal airway into the nasal passage by allowing the sidewall of the cannula to flex and bend as it is navigated through the internal anatomy of the nasal passage.

As shown best in FIGS. 10-12, the holes 50 can be located in the elongate sides of the cannula 18. Although illustrated as several rows of diamond-shaped holes, it will be appreciated that other arrangements and shapes are possible. For example, the holes may be circular, rectangular or another shape, and the cannula may include more than or less than three rows of holes.

In the embodiment of FIGS. 10-17, conduits 52 are embedded in the sidewall 24 of the cannula by inserting the conduits into respective channels 54 in the sidewall. The conduits may be formed from a flexible material so as to facilitate the insertion, removal and/or replacement of the conduits in the channels. The conduits may be made from a biocompatible extruded silicone tubing, for example, or another suitable material. The conduits may have an outer diameter of about 0.010 inches-about 0.150 inches and preferably about 0.100 inches, and an inner diameter of about 0.030 inches-about 0.080 inches and preferably about 0.060 inches. The conduits can be built as part of the lumen rather than inserting a cannula or tube into the channels.

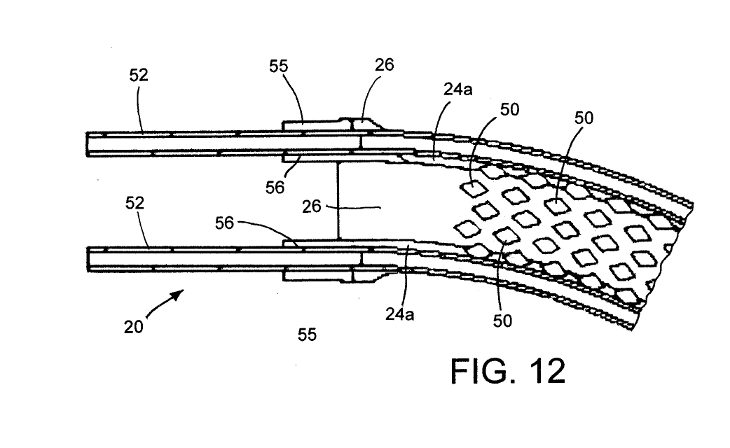

The channels 54 can extend along the entire length of the cannula 18. For example, as shown in FIGS. 10-14, the channels extend along the cannula 18 from the flange 26 to the distal end 22 of the nasopharyngeal airway 10. As shown best in FIG. 12, the flange has a pair of extensions 55 that surround through passages 56. The through passages 56 provide a passageway from the proximal end of the nasopharyngeal airway to the channels, which extend from a distal side of the flange to the distal end of the nasopharyngeal airway. Each conduit can be inserted into a respective through passage 56 and into the channel. Although the illustrated embodiment shows the channels extending to the tip 23, other configurations are possible. For example, the channels may stop short of the tip where the channels merge with the lumen through channel openings in the sidewall of the cannula or any part of the nasopharyngeal tube.

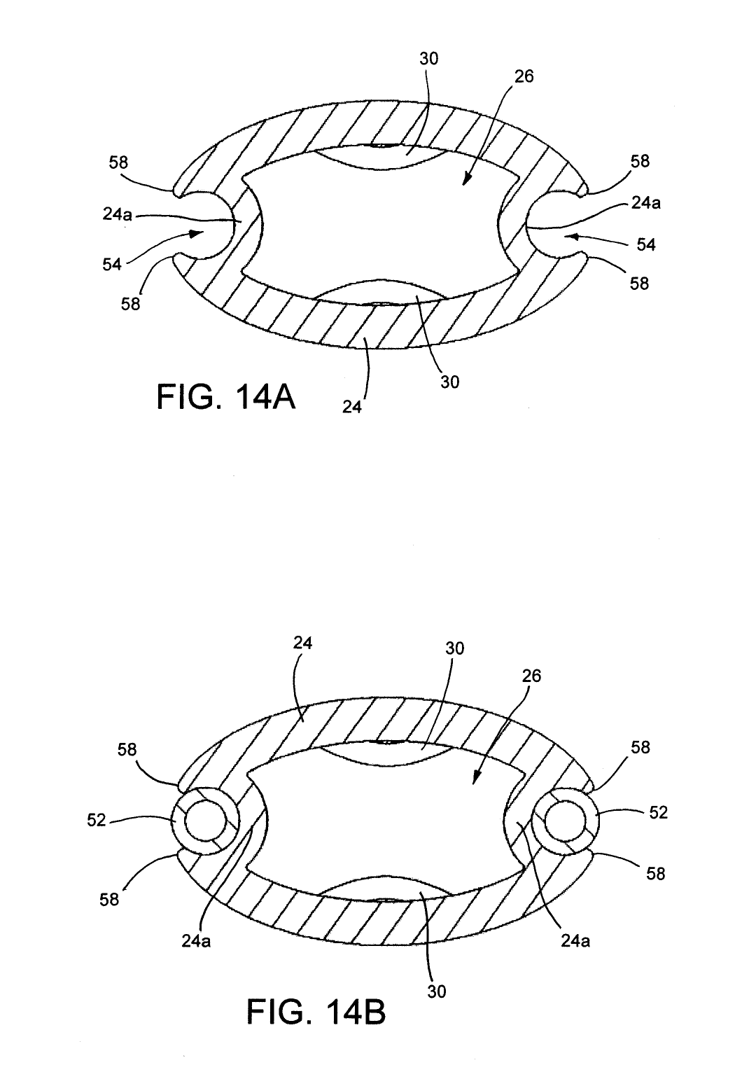

The channels 54 may be shaped to releasably hold the conduits 52. An exemplary embodiment of the profile of the channels is shown in FIGS. 14A and 14B. In FIG. 14A, the channels are shown without the conduits inserted therein and in FIG. 14B, the conduits are inserted into the channels. The channels may have a “C” shape with outer edges 58 that abut against the conduit when the conduit is inserted into the channel (FIG. 14B). The edges also can protect the conduits from being pulled, snagged and/or tangled, etc. while the nasopharyngeal airway is being inserted into the nasal passageway.

The space between the edges 58 of each respective channel 54 provides a space into which the conduit 52 can be inserted to embed the conduit in the sidewall. For example, the space between the edges can be wide enough so as to allow the conduit to be pressed into the channel, but narrow enough that the edges limit movement of the conduit in the channel and inhibit removal of the conduit from the channel. The conduit and/or the channel may be constructed from a resiliently deflectable material that allows for a temporary deflection and/or deformation during the insertion of the conduit in the channel. Upon insertion of the conduit in the channel, the structures may naturally return to their original shape, thereby holding the conduit in the channel. The conduit can be removed by the channel in a similar manner, for example, by pulling the conduit out of the channel and/or by flexing the channel and/or conduit.

Although illustrated as an open channel 54 that extends the length of the cannula 18, it will be appreciated that the edges 58 may include one or more retaining features to hold the conduits in the channels. For example, the edges of each channel can be connected to one another at spaced apart locations along the length of the cannula to reinforce the retention of the conduit in the channel. Additionally or alternatively, other retaining elements or features can be utilized. The retaining elements may form a generally smooth transition between the edges of the channel and the conduit so as to avoid causing trauma when the nasopharyngeal airway 10 is slid into the nasal passage.

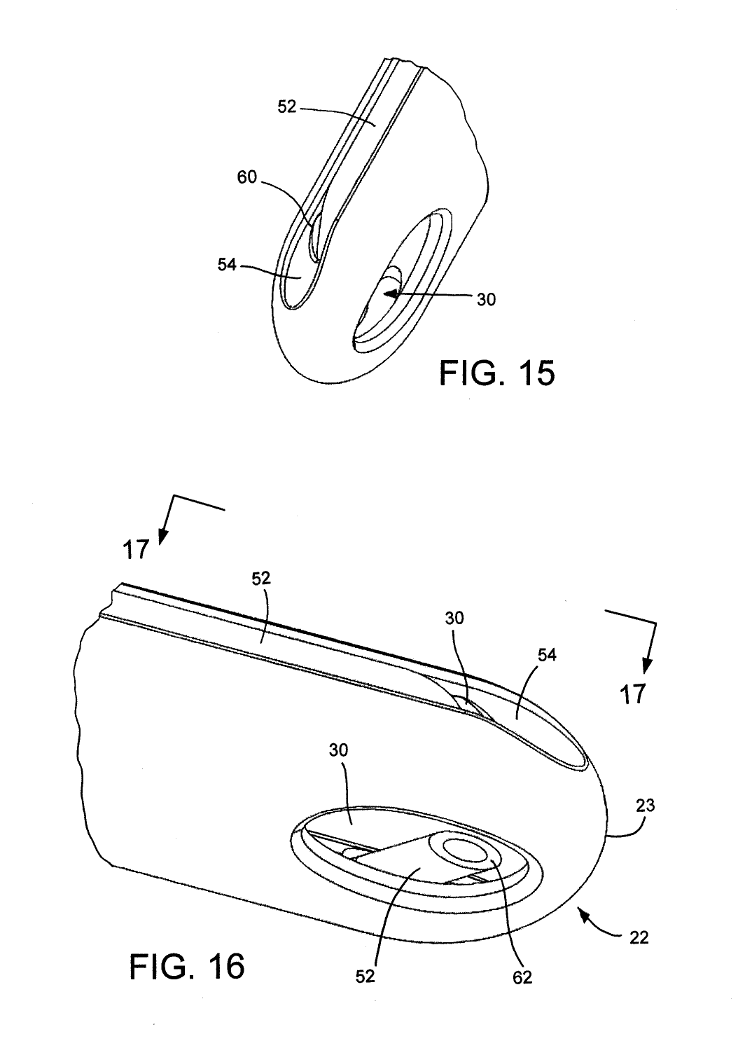

Referring now to FIGS. 15-17, the sidewall 24 of the cannula includes a pair of channel openings 60 that provide a pathway from the channels 54 to the lumen 26. Respective distal ends 62 of the conduits 52 can be inserted through the channel openings 60 when the conduits are inserted into the channels, thereby providing fluid communication between the conduits and the lumen. The channel openings also may be configured to retain the conduits in the channels, for example, the conduits can be press fit into the openings such that the outer wall of the conduits is frictionally retained by the channel openings. Alternatively, the conduit can be held by glue or another retaining implement. The connection between the conduit and the opening to the lumen can reduce the likelihood of the conduit from being snagged, tangled or pulled when inserting the nasopharyngeal airway into the nasal passage. Additionally, as discussed above, the diameter of the distal ends 62 of the conduits may be greater than the diameter of the conduits at a location proximal to the distal end of the conduit so as to reduce the likelihood of the conduit becoming blocked should debris enter the lumen.

Although the drawings and description are directed to one or more embodiments, equivalents and modifications will occur to others skilled in the art upon the reading and understanding of the specification.

Features that are described and/or illustrated with respect to one embodiment may be used in the same way, or in a similar way, in one or more other embodiments and/or in combination with, or instead of, the features of the other embodiments.

It is understood that equivalents and modifications to the foregoing embodiments will occur to others skilled in the art upon the reading and understanding of the specification.

What is claimed is:

1. A nasopharyngeal airway for providing an air passage through a nose to a nasopharynx of a patient comprising:
a cannula having a proximal open end, a distal closed end, and a sidewall surrounding a lumen of the cannula;
an opening in the sidewall at a distal portion of the cannula for providing fluid communication between a distal portion of the lumen and the nasopharynx when the cannula is inserted into the patient; and
a conduit in the sidewall of the cannula, the conduit having an opening adjacent to the proximal open end of the cannula and a conduit opening in a distal portion of the cannula for providing fluid communication between the conduit and the distal portion of the lumen.

2. The nasopharyngeal airway of claim 1, wherein the conduit opening is closer to the distal closed end of the cannula than it is to the proximal open end of the cannula.

3. The nasopharyngeal airway of claim 1, wherein an outer perimeter of the cannula is an oval or elliptical shape.

4. The nasopharyngeal airway of claim 3, wherein the oval or elliptical shape has a major axis and a minor axis, and wherein the conduit opening is on the major axis and the sidewall opening is on the minor axis.

5. The nasopharyngeal airway of claim 1, further comprising a second conduit in the sidewall of the cannula, the second conduit having an opening adjacent to the proximal open end of the cannula and a conduit opening for providing fluid communication between the second conduit and the lumen.

6. The nasopharyngeal airway of claim 5, wherein the conduits are parallel to one another.

7. The nasopharyngeal airway of claim 5, wherein at least one of the conduit openings is elongate such that the conduit opening has a cross-sectional area that is greater than a cross-sectional area of the conduit proximal to the conduit opening.

8. The nasopharyngeal airway of claim 1, wherein the conduit opening is perpendicular to the opening in the sidewall of the cannula.

9. The nasopharyngeal airway of claim 1, wherein the distal closed end of the tubular member is tapered from the opening in the sidewall to a tip of the distal closed end.

10. The nasopharyngeal airway of claim 9, wherein the tip of the distal closed end is round.

11. The nasopharyngeal airway of claim 1, further comprising a second opening in the sidewall at a distal portion of the cannula for providing fluid connection between the distal portion of the lumen and the nasopharynx when the cannula is inserted into the nose

12. The nasopharyngeal airway of claim 1, further comprising a flange extending laterally from the proximal open end of the cannula, wherein when the cannula is inserted into the nose, the flange abuts a nostril opening to limit a depth to which the cannula can be inserted into the nose.

13. The nasopharyngeal airway of claim 12, wherein the flange has a width dimension that is greater than a height dimension.

14. The nasopharyngeal airway of claim 1, wherein the conduit is integral with the sidewall of the cannula.

15. The nasopharyngeal airway of claim 1, wherein the conduit is inserted into a channel in the sidewall of the cannula, the channel extending from the proximal open end to a channel opening connecting the channel to the lumen.

16. The nasopharyngeal airway of claim 1, further comprising a plurality of holes through the sidewall of the cannula, the holes providing a pathway from the lumen through the sidewall of the cannula to a turbinate and/or septum of the patient when the nasopharyngeal airway is inserted into the nose.

17. The nasopharyngeal airway of claim 16, wherein the holes are configured to face the turbinate and/or septum of the patient to provide physiologic humidification and warming of airflow as air is drawn through the lumen to the nasopharyngeal airway and into the lungs.

18. A nasopharyngeal airway comprising:
a tubular member having a closed distal end, a sidewall surrounding a lumen, and an opening in the sidewall for providing fluid communication between a distal portion of the lumen and a nasopharynx of a person when the tubular member is inserted into a nose, wherein the tubular member is tapered from the opening in the sidewall to the closed distal end to facilitate the insertion of the tubular member into the nose; and
a pair of parallel conduits in the sidewall of the tubular member, each conduit in fluid communication with the distal portion of the lumen through respective conduit openings.

19. The nasopharyngeal airway of claim 18, further comprising a second opening in the sidewall that allows fluid communication between a distal portion of the lumen and a nasopharynx of a person when the tubular member is inserted into a nose.

20. The nasopharyngeal airway of claim 18, wherein the opening of each conduit to the lumen has a cross-sectional area that is greater than the cross-sectional area of the conduit proximal to the conduit opening.

21. The nasopharyngeal airway of claim 18, wherein the conduits are integrally formed in the sidewall of the cannula.

22. The nasopharyngeal airway of claim 18, wherein the conduits are inserted into channels in the sidewall of the cannula.

23. The nasopharyngeal airway of claim 18, further comprising a plurality of holes through the sidewall of the cannula, the holes providing a pathway from the lumen through the sidewall of the cannula to a turbinate of the patient when the nasopharyngeal airway is inserted into the nose.

24. A tip for a nasopharyngeal airway comprising:
a tubular member having a sidewall surrounding a lumen;
a pair of conduit openings in the sidewall of the tubular member, each of the conduits in fluid communication with the lumen through the respective openings; and
a pair of sidewall openings in the sidewall of the tubular member for providing fluid communication between the lumen and a nasopharynx of a person when the tubular member is inserted into a nose;
wherein the conduit openings and the sidewall openings are circumferentially offset from one another.

25. The tip of claim 24, wherein the tubular member is tapered from a portion of the sidewall adjacent the conduit openings to a rounded tip to facilitate insertion of the tubular member into the nose.

26. The tip of claim 24, wherein the tubular member has an outer perimeter that is an elliptical shape having a major axis and a minor axis, and wherein the pair of conduit openings are on the major axis and the pair of sidewall openings are on the minor axis.
